(19) United States
(12) Patent Application Publication
(10) Pub. No.: US 2024/0197885 A1
(43) Pub. Date: Jun. 20, 2024

(54) Use of Self-Assembled Nanocrystals

(71) Applicants: Marie Paule PILENI, PARIS (FR); UNIVERSITE DE PARIS, PARIS (FR); CENTRE NATIONAL DE LA RECHERCHE SCIENTIFIQUE - CNRS, PARIS (FR)

(72) Inventor: Marie Paule Pileni, Paris (FR)

(21) Appl. No.: 18/013,764

(22) PCT Filed: Jun. 30, 2021
(86) PCT No.: PCT/FR2021/051200

§ 371 Date: Dec. 29, 2022

(30) Foreign Application Priority Data
Jul. 1, 2020 (FR) 2006967

(51) Int. Cl. A61K 41/00; A61K 9/51; A61K 33/242; A61K 33/26; A61P 35/00

(52) U.S. Cl. CPC A61K 41/0052; A61K 9/5123; A61K 33/242; A61K 33/26; A61P 35/00

(57) ABSTRACT

The present invention relates to a self-assembled structure for use in the treatment of mammalian cancer, the structure being between 50 and 1,500 nm in size and comprising a plurality of nanocrystals chosen from metal or metal oxide nanocrystals, a plurality of ligands being covalently linked to the surface of each of the nanocrystals, the ligands comprising hydrocarbon chains. The invention also relates to an injectable composition comprising said structure.

### TECHNICAL FIELD

The invention relates to self-assembled structure formed from nanocrystals for use in the diagnosis or treatment of a variety of diseases and disorders, such as cancer, in order to introduce the said structures parenterally into a mammal to create localized hyperthermia in a target area.

### PRIOR ART

Radiation therapy is often a component of the multidisciplinary approach to the treatment of many tumors. However, as a single modality, radiation therapy is not able to eradicate all regional recurrences and/or to cure localized cancers.

One of the promising applications in medicine is based on the use of nanometric agents for the implementation of spatially and temporally controlled hyperthermia or photothermal therapy (PTT). This type of therapy has been intensively studied over the last decades. It represents an adjuvant or neo-adjuvant strategy that can be used in the treatment of chemo-resistant and difficult-to-treat tumors. It is known that malignant tissues are destroyed above 42-43° C.

In this technique, the main goal is to use nanoparticles that can be remotely activated by an external stimulus, such as a laser or an alternating magnetic field, this in order to generate controlled hyperthermia in the target region. In particular, in the case of photothermal therapy, we are dealing with a minimally invasive hyperthermia treatment based on the conversion of optical energy into thermal energy by nanoparticles while under near-infrared laser irradiation.

Currently, there is a wide range of photoactivatable nanomaterials including metallic plasmonic nanoparticles (such as gold nanoparticles), carbon absorbing nanomaterials (such as carbon nanotubes, graphene nanotubes and analogues) or iron oxide nanoparticles. In order to select a suitable nanomaterial as a phototherapeutic agent, in addition to its light absorption capabilities, other parameters need to be taken into consideration, such as low toxicity, ease of preparation and scale-up, favorable bio-distribution in mammals and an in vivo life cycle.

In this sense, iron oxide nanoparticles are particularly interesting due to their weak toxicity, natural metabolism by endogenous mammalian proteins and the clinical authorization of certain formulations. They could therefore be used in the treatment of cancers, or other diseases in mammals for which photothermal therapy would be effective.

### Technical Issue

One of the limitations of the use of isolated nanocrystals is nonetheless their small size, which does not allow an efficient targeting of the tissue to be treated or rather may indeed lead, after injection, to a distribution of the photothermal therapy agents outside the target cells.

The present inventors have developed a new photothermal therapy agent that allows its use in the imaging diagnosis or treatment of cancer or other diseases, such as cardiovascular diseases or rheumatism, while at the same time ensuring a good internalization in the target cells of the said agent and avoiding their accumulation in the liver and other organs.

### DESCRIPTION OF THE INVENTION

To this end, according to a first aspect, the invention relates to a self-assembled structure for use in the treatment of mammalian cancer, the said structure having a size of 50 to 1,500 nm, preferably 50 to 800 nm and comprises a plurality of nanocrystals selected from metal or metal oxide nanocrystals, a plurality of ligands covalently bound to the surface of each of the said nanocrystals.

According to a second aspect, the invention relates to an injectable composition comprising self-assembled structures according to the first aspect and a pharmaceutically acceptable excipient or diluent. The presence of an excipient or diluent makes the composition biocompatible. The said pharmaceutically acceptable diluent is preferably saline solution.

According to a third aspect, the invention relates to a self-assembled structure according to the first aspect or a composition according to the second aspect for use in a method for diagnosing cancer in a mammal by means of imaging, such as computed tomography (CT) or magnetic resonance imaging (MRI). Due to the significantly larger size compared to unassembled nanocrystals, these structures can be easily spotted by imaging.

According to a fourth aspect, the invention relates to a self-assembled structure according to the first aspect or a composition according to the second aspect for use in a method inducing cellular destruction of cancerous cells in a mammal by photothermal and/or magnetothermal treatment comprising contact of the cancerous cells with a plurality of the said structures and irradiation thereof with infrared light, respectively application of a magnetic field.

According to a fifth aspect, the invention relates to a self-assembled structure according to the first aspect or a composition according to the second aspect for use in a method of inducing cellular destruction of cancerous cells in a mammal by radiation therapy comprising contact of the cancerous cells with a plurality of the said structures and administration of a dose of radiation therapy.

By combining the localization of structures after injection by means of medical imaging and the induction of cellular destruction of the cells containing the structures using photothermal therapy, access to inaccessible areas of the body is promoted, thereby allowing the development of personalized medicine.

Another major interest of the invention is that it can be used both for in vivo applications, as explained below, and for in vitro applications. For example, during biopsy of a tumor, cancerous cells can be detected as a function of the degree of internalization of the structures by these cells.

### Definitions

The term “cancer” refers to any pathological condition that is typically characterized by an unregulated growth of cells. By way of examples of cancer, one can include carcinomas, lymphomas, blastomas, sarcomas and leukemias, and more specifically squamous cell lung carcinomas, colon adenocarcinomas, mesotheliomas, gliomas, breast adenocarcinomas, melanomas, clear cell renal cell carcinomas, prostate cancer, hepatocarcinomas and multiple myelomas.

The term “treatment” refers to any prophylactic or suppressive therapeutic measure for a disease or disorder that results in a desirable clinical effect or any beneficial effect, including but not limited to the elimination or reduction of one or more symptoms, regression, slowing or cessation of progression of the cancer or related disorder.

The term “apoptosis” refers to the programmed death of cells.

The term “necrosis” refers to the abnormal, unscheduled death of a cell or tissue. It therefore contrasts with apoptosis.

### DETAILED DESCRIPTION OF THE INVENTION

As indicated, according to a first aspect, the invention relates to a self-assembled structure for use in the treatment of mammalian cancer, the said structure having a size of 50 to 1,500 nm, preferably 50 to 800 nm and comprises a plurality of nanocrystals selected from metal or metal oxide nanocrystals, a plurality of ligands covalently bound to the surface of each of the said nanocrystals.

The nanocrystals used have their own properties when isolated. When they are assembled, they have collective properties specific to the assembly due either to dipolar interactions or to intrinsic properties due to the assemblies. Thus, when the nanocrystals are in the form of a self-assembled structure according to the invention, these structures behave as nanoradiators, whereas this property does not exist for isolated nanocrystals. Water plays a major role on the nanoradiator without it being known why. In the case of ferrites, after 8 days of internalization, disordered “clusters” are formed with an onset of degradation, whereas in the systems of the invention, the assembly is maintained and consequently they can keep their collective properties.

By specifically targeting diseased sites, toxicity on other parts of the organism is greatly reduced. The quantity of self-assembled structures to be used is thus reduced because the majority is concentrated towards the targeted site.

These structures are all the more interesting as they do not need a complementary carrier during their synthesis or use. They are therefore free standing, which allows a greater freedom of manipulation and functionalization.

Such a self-assembled structure exhibits a size of 50 to 1,500 nm, preferably 50 to 800 nm, most preferably between 55 and 600 nm. The size of the structures is measured from the images obtained using transmission electron microscopy (TEM).

The structure corresponds to an assembly of a plurality of nanocrystals selected from metal or metal oxide nanocrystals.

According to one embodiment, the nanocrystals are selected from metal nanocrystals such as gold, silver, iron, cobalt, zinc, copper, aluminum, chromium, silicon and selenium nanocrystals. Preferably, the metal nanocrystals are gold nanocrystals.

Moreover, according to an alternative embodiment, the units forming the assembled structures may be carbon nanotubes.

According to an alternative or complementary embodiment, the nanocrystals are selected from metal oxide nanocrystals such as iron oxide, cobalt oxide, silica, manganese oxide, nickel oxide, vanadium oxide, chromium oxide, titanium oxide. Preferably, the said nanocrystals are selected from iron oxides such as ferrites, including in particular magnetite (Fe₃O₄), maghemite (y-Fe₂O₃). Alternatively, graphene can be used.

More preferably, the said metal oxide nanocrystals are in the form of a mixture of magnetite (Fe₃O₄) and maghemite (y-Fe₂O₃), the two species coexisting within the mixture. This is due to the fact that part of the Fe Il oxidizes to Fe III, leading to a heterogeneous mixture of iron oxides in which the iron is in its Fe Il or Fe Ill form. The use of the said ferrites is therefore advantageous because they impart magnetic properties to the self-assembled structure.

According to a preferred embodiment, the self-assembled structures are formed on the basis of a mixture of metal nanocrystals and metal oxide nanocrystals, as indicated above.

More preferably, when the self-assembled structures are formed on the basis of a mixture of metal nanocrystals and metal oxide nanocrystals, the said mixture is a mixture of gold nanocrystals and iron oxide nanocrystals of the chemical formula Fe₃O₄ and y-Fe₂O₃.

According to a preferred alternative embodiment, when the self-assembled structures are formed on the basis of the metal oxide nanocrystals alone, the metal oxide nanocrystals are iron oxide nanocrystals of chemical formula Fe₃O₄ and y-Fe₂O₃.

The term “ligand” refers to a molecule carrying at least one chemical function allowing it to bind to one or a plurality of atoms or ions forming the nanocrystals. The person skilled in the art will know how to choose the nature of the chemical function depending on the nature of the nanocrystals. These chemical functions may be selected from functional groups containing at least one heteroatom, such as nitrogen, sulfur, phosphorus, silicon or oxygen.

According to one embodiment, the ligands are covalently bound to the surface of each of the metal nanocrystals through a functional group selected from sulfanyl, carboxyl and hydroxyl groups.

The surface of the nanocrystals is thus modified so as to make them capable of self-assembly. Thus, a plurality of ligands is covalently bound to the surface of the said nanocrystals. The said ligands comprise hydrocarbon chains carrying at least one functional group or are polymeric in nature.

These hydrocarbon chains have from 4 to 36 carbon atoms, preferably from 12 to 18 carbon atoms, and are covalently bound to the surface of each of the nanocrystals by the said functional group selected from sulfanyl, carboxyl and hydroxyl groups.

The ligands used to functionalize the nanocrystals are selected from:
the fatty acids comprising from 4 to 36 carbon atoms, preferably from 12 to 18 carbon atoms. Preferably, the fatty acid is oleic acid.
the polyesters (in particular polylactide, polyglycolide and their poly[lactide-co-glycolide] copolymers), poly(alkyl cyanoacrylate) (PACA), as well as poly(amino acid)s (PAA), or polypeptides;
the alkyl thiols comprising from 4 to 36 carbon atoms, preferably from 12 to 18 carbon atoms. Preferably, the alkyl thiol is octadecanethiol and dodecanethiol and/or
the fatty alcohols comprising from 4 to 36 carbon atoms, preferably from 12 to 18 carbon atoms.

According to one embodiment, the nanocrystals have a plurality of surfactants adsorbed to their surfaces, the said surfactants being selected from phospholipids, such as distearoylphosphatidylcholine, distearoylphosphatidylethanolamine, dipalmitoylphosphatidylcholine, and dipalmitoylphosphatidylethanolamine; the halogenated cationic compounds having a chain comprising 12 to 16 carbon atoms such as cetylpyridinium chloride, cetylpyridinium bromide, cetyltrimethylammonium bromide, cetyltrimethylammonium chloride, cetyldimethylethylammonium bromide, cetylbenzyldimethylammonium chloride, cetyltributylphosphonium bromide, dodecyltrimethylammonium bromide and tetradecyltrimethylammonium bromide.

The term “surfactant” is equivalent to the term “surface active agent”.

Preferably, the surfactant is dodecyltrimethylammonium bromide (also known as DTAB).

The hydrocarbon chains of the surfactants and ligands which are present on the surface of the nanocrystals exhibit Van der Waals type interactions, with the surfactant chains “slotting” in the spaces formed by those of the said ligands.

The presence of the surfactants therefore imparts a hydrophilic character to the structures and allows the solubilization of the nanocrystals coated with the hydrocarbon chains in an aqueous medium.

According to one embodiment, the nanocrystals have a plurality of aliphatic hydrocarbons adsorbed to their surfaces, the said aliphatic hydrocarbons being selected from hydrocarbons having from 16 to 36 carbon atoms. As in the case of surfactants, their hydrocarbon chains “slot” between the hydrocarbon chains of the ligands and surfactants. The presence of these aliphatic hydrocarbons provides a degree of flexibility to the structure and allows for varied architectures to be obtained.

Preferably, the hydrocarbon chains of the said hydrocarbons adsorbed on the surface have less than three unsaturations, preferably less than two unsaturations, more preferably one unsaturation or having no unsaturation. This allows for an arrangement of chains extending radially around the constituent nanocrystals so as to minimize interaction between the ligand chains belonging to the same crystal. In the presence of hydrocarbon chains with few unsaturations and thanks to their radial arrangement, the space created between these chains of the same nano-crystal allows to “accommodate” hydrocarbon chains belonging to surfactants. In excess of three unsaturations, there is a risk of an overly fast degradation of the said chains.

The aliphatic hydrocarbons are selected from hydrocarbons having from 16 to 36 carbon atoms, preferably from 16 to 24 carbon atoms. Preferably, these hydrocarbons may be selected from dodecane C₁₂H₂₆ and octadecane C₁₈H₃₈. Preferably, such an aliphatic hydrocarbon is octadecene (C₁₈H₃₆).

According to one embodiment, the structure is in the form of a capsule comprising a central volume surrounded by a hollow shell formed by nanocrystals; the said central volume being empty or partially filled by the said nanocrystals. The presence of aliphatic hydrocarbons as described above is essential for the formation of the said capsules.

Preferably, the shell is formed by metal oxide nanocrystals and the central volume is partially filled with a mixture of gold nanocrystals and metal oxide nanocrystals.

In a more detailed manner, when the nanocrystals are functionalized with the above-described ligands, and when surfactants and aliphatic hydrocarbons are adsorbed on their surface, the self-assembled structure takes the form of a capsule comprising a central volume surrounded by a hollow shell formed by the said nanocrystals.

In particular, the hollow shell is formed from a plurality of layers of functionalized nanocrystals, the hydrocarbon chains of ligands of which are confined within the layers.

Preferably, the nanocrystals forming the shell are metal oxide nanocrystals. Even more preferably, they are nanocrystals selected from iron oxides such as ferrites, including in particular magnetite (Fe₃O₄), maghemite (y-Fe₂O₃).

According to one embodiment, the said central volume is empty. This embodiment is illustrated by the self-assembled structures of type A, the preparation of which is described here below.

According to an alternative embodiment, the said volume is partially filled with the said functionalized nanocrystals and with surfactants and aliphatic hydrocarbons adsorbed on their surfaces, as described above. The said central volume is filled with the said nanocrystals at a filling rate between 10 to 50% of the volume of nanocrystals relative to the total central volume. This embodiment is illustrated by the self-assembled structures of types C, D and E whose preparation is described below.

The nanocrystals filling the central volume of the capsule are metal oxide nanocrystals or a mixture of metal and metal oxide nanocrystals. Preferably, the metal nanocrystals are gold nanocrystals and the metal oxide nanocrystals are selected from iron oxides such as ferrites, including in particular magnetite (Fe₃O₄), maghemite (y-Fe₂O₃).

The filling rate of the central volume is measured from the images obtained by transmission electron microscopy.

Furthermore, the central volume of the self-assembled structure may additionally contain therapeutically targeted molecules or peptides for localizing specific receptors present in the body of a diseased mammal, for example, receptors specific to cancerous cells in the body of a mammal suffering from cancer. Preferably, such therapeutically targeted molecules placed within the central volume of the shell have a hydrophobic moiety capable of associating with the said shell in their structures.

According to another embodiment, the structure exhibits a crystalline-type architecture within which the nanocrystals are organized in a face-centered cubic configuration. This compact structure is obtained in the absence of aliphatic hydrocarbons. Preferably, the nanocrystals are iron metal oxide nanocrystals of the chemical formula Fe₃O₄ and y-Fe₂O₃.

This embodiment is illustrated by the self-assembled structures of type B, the preparation of which is described below.

The absence of aliphatic hydrocarbons adsorbed on the surfaces of the nanocrystals no longer allows a capsule composed of a shell surrounding a central volume to be obtained. The obtained structure is compact.

In these structures, the size of the nanocrystals is between 2 and 12 nm, preferably between 3 and 10 nm, more preferably between 6 and 10 nm.

The size distribution of the nanocrystals is less than 10%. It is calculated by measuring the size of 500 to 1,000 nanocrystals, preferably about 500 nanocrystals, from the TEM images and calculating the average.

By way of example, different possible structures, structures A to E, corresponding to the embodiments described above, are described below with their method of preparation.

According to a second aspect, the invention relates to an injectable composition comprising self-assembled structures according to the first aspect and a pharmaceutically acceptable excipient or diluent. The presence of an excipient or a diluent makes the composition biocompatible and promotes its introduction into the body of a mammal. Any type of excipient or diluent that can be used for injectable compositions is suitable. By way of example of diluents, one can name lactose, maltose, sucrose, sorbitol, mannitol, other derivatives ending in ose and oxide or a mixture thereof. By way of examples of excipients one can name water, propylene glycol, glycerin or a mixture thereof.

Preferably, the composition is aqueous.

The nanocrystals coated with the ligands, as described above, are hydrophobic in nature whereas the excipient or the diluent is not fully hydrophobic. The presence of a plurality of the surfactants on the surface of these nanocrystals renders the said self-assembled structure soluble in the said pharmaceutically acceptable excipient or diluent after self-assembly.

Preferably, the said pharmaceutically acceptable diluent that is present in the composition is water or saline solution.

Thus, preferably, the invention relates to an injectable composition comprising self-assembled structures according to the first aspect and an excipient or saline solution.

The composition of a saline solution is well known to the person skilled in the art. By way of example, saline solution is generally composed of distilled water and sodium chloride (NaCl) diluted 9 per mille (which is to say, a solution of 0.9% mass/volume of NaCl, which is to say, 9 g L-1). It contains around 154 mEq/l (which is to say, 0.154 mol/L) of sodium and chloride ions.

The invention therefore relates to the self-assembled structure as described herein or to the composition comprising a plurality of the said structures for their use in the treatment of cancer in mammals, wherein the cancer is a tumor/metastasis. The said structure or composition can therefore be used in the treatment of breast, lung, prostate, intestinal, cartilage, lymph node, spleen, liver, brain, esophagus, stomach, cervical and melanoma cancer.

Preferably, the said self-assembled structure according to the first aspect or the said composition according to the second aspect is intended to be used in the treatment of cancer, rheumatism or cardiovascular diseases.

These structures can therefore be used in a method inducing cellular destruction of cancerous cells in a mammal by photothermal and/or magnetothermal treatment comprising contact of the cancerous cells with a plurality of the said structures and irradiation thereof with infrared light, or respectively the application of a magnetic field. The cell destruction takes place by necrosis and/or apoptosis.

According to a third aspect, the invention relates to a self-assembled structure according to the first aspect or a composition according to the second aspect for use in a method for diagnosing cancer in a mammal by imaging, such as computed tomography (CT) or magnetic resonance imaging (MRI). Due to the significantly larger size compared to unassembled nanocrystals, these structures can easily be spotted by imaging.

Indeed, the particular magnetic properties of iron oxides make them usable in medical imaging by magnetic resonance (MRI). Iron oxides are superparamagnetic and therefore, as soon as a magnetic field is removed, they become disoriented. The relatively weak contrast of isolated nanocrystals makes their use in medical imaging difficult. On the other hand, in the case of self-assembled structures starting from nanocrystals in a cellular medium, under the action of a magnetic field, they try to align themselves according to the field that has been applied and thus become good candidates for medical imaging. Moreover, the alignment of these structures allows their displacement within the biological environment.

The iron oxide crystals are in the form of magnetite (Fe₃O₄) or maghemite (y-Fe₂O₃) and are therefore composed of Fe₂+, Fe₃+ ions.

According to a fourth aspect, the invention relates to a self-assembled structure according to the first aspect, a composition according to the second aspect, for use in a method of inducing cellular destruction of cancerous cells in a mammal by photothermal and/or magnetothermal treatment comprising contact of the cancerous cells with a plurality of the said structures and irradiation thereof with infrared light, or respectively the application of a magnetic field.

Thus, the structures or the compositions containing the structures are injected directly into the cancerous tissues; then a laser beam and/or a radiofrequency field is applied and the cells having internalized the said structures are locally destroyed.

Indeed, in the presence of a high frequency magnetic field (105 to 106 Hz) the iron oxide nanoparticles heat up locally, therefore inducing the appearance of localized hyperthermia.

According to a fifth aspect, the invention relates to a self-assembled structure according to the first aspect or a composition according to the second aspect for use in a method of inducing cellular destruction of cancerous cells in a mammal by radiation therapy comprising contact of the cancerous cells with a plurality of the said structures and the administration of a dose of radiation therapy.

Indeed, the administration of a dose of radiation therapy targeting cancerous cells that have incorporated the said structures enables a high dose of energy to be emitted locally. This effect is thus potentiated by the self-assembled structures.

### BRIEF DESCRIPTION OF THE DRAWINGS

Other features, details and advantages of the invention will become apparent from the detailed description below, and from an analysis of the appended figures, in which:

FIG. 1 shows an image obtained by transmission electron microscopy of a self-assembled structure A according to one embodiment of the invention;

FIG. 2 shows an image obtained by transmission electron microscopy of a self-assembled structure B according to one embodiment of the invention. The image obtained by X-ray diffraction shows that the nanocrystals are self-assembled in a face-centered cubic lattice.

FIG. 3 shows an image obtained by transmission electron microscopy of a self-assembled structure C according to one embodiment of the invention;

FIG. 4 shows an image obtained by transmission electron microscopy of a self-assembled structure D according to one embodiment of the invention;

FIG. 5 shows three images obtained by transmission electron microscopy of a self-assembled structure E according to one embodiment of the invention;

FIG. 6 shows two images obtained by electron microscopy of cells incubated after 8 days with self-assembled structures A (left) and B (right);

FIG. 7 shows a graph showing the release of heat when cells in which different structures are internalized for 24 hours with the same quantity of iron are subjected to overall laser irradiation for 5 minutes;

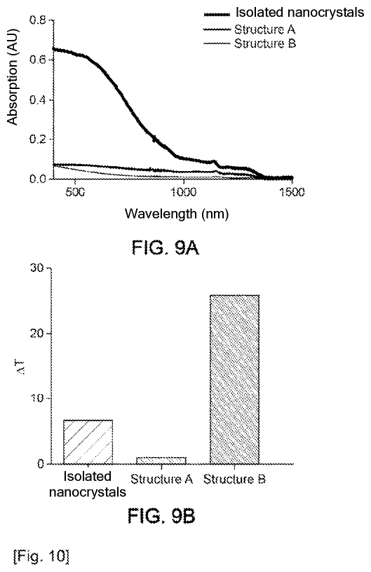

FIG. 8 shows the results of the damage caused by two different approaches demonstrating the importance of the local effect on necrosis and apoptosis;

FIG. 9 shows (a) the absorption spectra of ferrite nanocrystals dispersed in water and structures A and structures B at the same iron concentrations; (b) temperature elevation as a function of A and structures B, obtained by measuring temperature as a function of time;

FIG. 10 shows a graph showing the temperature rise as a function of the quantity of self-assembled gold nanocrystals and electron microscopy images of the structures tested;

FIG. 11 shows a graph showing the results of temperature rise for assemblies of type C. The left column represents crystalline assemblies of single gold nanocrystals organized in an FCC (face-centered cubic) crystal system and the right column those of the binary system (Fe₃O₄)Au₁₃;

FIG. 12 shows electron microscopy images of a tendon (a, b, c, d) on which a solution of structures A was deposited overnight (about 12 hours) at 4° C. before (a and b) and after 10 minutes (c, d) of irradiation. Optical microscopy image of a control tendon (e) and after irradiation of structures A (f). The arrows indicate the presence of holes. Histogram (g) of the average area of holes induced by structures A;

FIG. 13 shows electron microscopy images of structures B deposited on a tendon incubated overnight at 4° C. before (a) and after (b, c, d) irradiation (808 nm/1 W/cm²/10 min);

FIG. 14 shows images of mice into which were injected structures D, which is to say, a shell of ferrite nanocrystals containing a structure assembly of the NaZn₁₃ type, which is to say, (Fe₃O₄)Au₁₃. The results are reproducible. The first series (top) shows that after 4 months (bottom) the assemblies remain localized near the injection site.

FIG. 15 shows two graphs presenting the level of collagen signal (a) and the variation of collagen fiber length (b) in the presence of unassembled nanocrystals, of the structures D and B compared to the length of untreated fibers (control).

FIG. 16 shows microscopy images of structures D after 5 minutes irradiation (b) or 12 minutes irradiation (c) compared to the initial non-irradiated structures (a).

The following figures and description may therefore not only serve to further the understanding of the present invention, but also contribute to its definition, where appropriate.

The self-assembled structures used according to the invention exhibit several types of internal architectures, as described below. However, regardless of their composition, the one thing in common of these structures is that they are capable of self-assembly starting from nanocrystals.

### Methods of Preparation of Nanocrystals Constituting Self-Assembled Structures

Nanocrystals based on iron or gold oxides are used to form the self-assembled structures presented in detail below.

### Synthesis of Nanocrystals:

### Synthesis of Type 1 Iron Oxide Nanocrystals Comprising Octadecene

Step 1. Iron oleate precursors are prepared as follows: 10.8 g of iron (III) chloride, 36.5 g of sodium oleate, 40 mL of deionized water, 40 mL of ethanol, and 80 mL of hexane were mixed in a 500 mL three-necked flask. The mixture was brought to reflux at 60° C. for four hours. The dark red-black iron oleate precursors are dissolved in 100 ml of hexane; the hexane solution is washed three times with hot deionized water (about 50° C.) and separated using a separating funnel. A viscous product is obtained by evaporating the hexane in a rotary evaporator.
Step 2. A mother precursor solution having a concentration of 0.5 mol/kg is prepared by adding 1.5 g of octadecene per gram of iron oleate.
Step 3. During the synthesis of size 6.5 nm or 10 nm Fe₃O₄ nanocrystals, 4.8 g of precursor solution is mixed with 6.0 g of octyl ether and with 0.76 g of oleic acid when size 6.5 nm nanocrystals are used or with 0.365 g of oleic acid when size 10 nm nanocrystals are used. The mixture is heated to 110° C. and kept at this temperature for 60 minutes under nitrogen. Then, the solution is brought to boiling point (around 295° C.) and is kept at this temperature for 30 minutes. Then, the heat source is removed. The obtained colloidal solution is washed 5 times with an isopropyl alcohol/hexane mixture (1:1 v/v) by sedimentation and redispersion by centrifugation (5,000 rpm for 10 minutes). Lastly, the Fe₃O₄ nanocrystals are weighed and redispersed in chloroform to obtain a desired concentration of nanocrystals.

The nanocrystals therefore exhibit a hydrophobic character.

### Synthesis of Type 2 Iron Oxide Nanocrystals not Comprising Octadecene

Step 1. The previously described step 1 for the synthesis of type 1 iron oxide nanocrystals is repeated in its entirety.
Step 2. During the synthesis of size 6.5 nm or 10 nm Fe₃O₄ nanocrystals, the viscous product obtained in step 1 is mixed with 0.76 g of oleic acid (or respectively with 0.365 g of oleic acid when 10 nm nanocrystals are used) and with 6.0 g of octyl ether. The mixture is heated to 110° C. and kept at this temperature for 60 minutes under nitrogen. Then, the solution is brought to boiling point (around 295° C.) and is kept at this temperature for 30 minutes. Then, the heat source is removed. The obtained colloidal solution is washed 5 times with an isopropyl alcohol/hexane mixture (1:1 v/v) by sedimentation and redispersion by centrifugation (5,000 rpm for 10 minutes). Lastly, the Fe₃O₄ nanocrystals are weighed and redispersed in chloroform to obtain a desired concentration of nanocrystals.

The nanocrystals therefore exhibit a hydrophobic character.

### Synthesis of Gold Nanocrystals

Gold nanocrystals of 3.5 nm or 5 nm diameter are synthesized. Two solutions are prepared. The first solution consists in dissolving 0.20 mmol of chloro(triphenylphosphine) gold (I) in 25 mL of toluene, to which 500 μL of dodecanethiol (DDT) is added. The second solution contains 5 mmol of t-butylamine borane dissolved in 2 mL of toluene. The first and second solutions are placed in a silicone oil bath at 100° C. and stirred for 10 minutes. Then the second solution is slowly injected into the first solution. The clear, colorless mixture becomes a brown and then dark purple-red solution within one minute. Said solution is kept at 100° C. for an additional 4 minutes, after which the oil bath is removed. The gold nanocrystals precipitate from the colloidal solution through addition of 10 ml of methanol. The supernatant is removed. The black precipitate is dried under nitrogen flow to remove the remaining solvent. The gold crystals are weighed and redispersed in chloroform to obtain a desired concentration of nanocrystals.

The nanocrystals therefore exhibit a hydrophobic character.

### Preparation Methods of the Self-Assembled Structures

A preferred method of synthesizing the nanocrystals constituting each of the self-assembled structures described above is described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500, a detailed example of which is shown in Example 1 below.

### Self-Assembled Structure of Type A

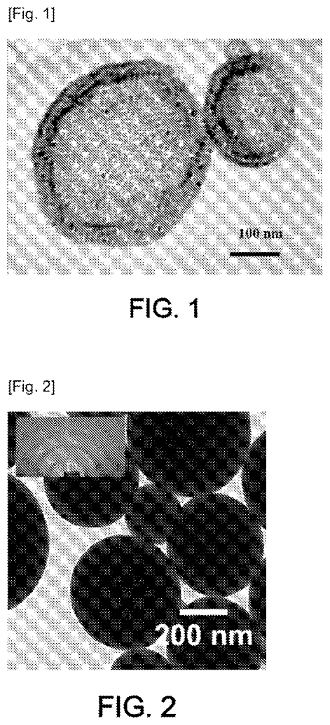

A first self-assembled structure according to the invention is in the form of an assembly of metal oxide nanocrystals. Preferably, the nanocrystals forming this assembly are in the form of a mixture with ferrites of chemical formula Fe₃O₄ and y-Fe₂O₃ as the basis. The nanocrystals used to obtain this structure are obtained according to the method of synthesis of type 1 iron oxide nanocrystals (of 6.5 nm or 10 nm) comprising an aliphatic hydrocarbon, for example octadecene. This type of assembly is hereinafter referred to as structure A and is illustrated in FIG. 1. Structure A, having a size between 70 and 270 nm, preferably between 100 and 200 nm, exhibits a 3D architecture in the form of a capsule, comprising a central volume surrounded by a hollow shell formed by the said nanocrystals. The said central volume is empty.

The synthesis of such structures A is described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500 and is illustrated in detail in example 2 below.

### Self-Assembled Structure of Type B

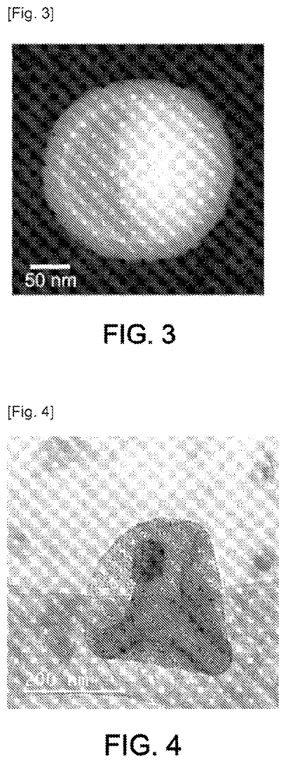

A second self-assembled structure used according to the invention is in the form of an assembly of metal oxide nanocrystals. Preferably, the nanocrystals forming this assembly are in the form of a mixture with ferrites of chemical formula Fe₃O₄ and y-Fe₂O₃ as the basis. The nanocrystals used to obtain this structure are obtained according to the method of synthesis of type 2 iron oxide nanocrystals (of 6.5 nm or 10 nm) not comprising a hydrocarbon such as octadecene. This type of assembly is hereinafter referred to as structure B and is illustrated in FIG. 2. Structure B, having a size between 55 and 85 nm, preferably between 60 and 80 nm, exhibits a spherical (3D) architecture of crystalline type within which the nanocrystals are organized in a face-centered cubic configuration.

The synthesis of such structures B is described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500.

### Self-Assembled Structure of Type C

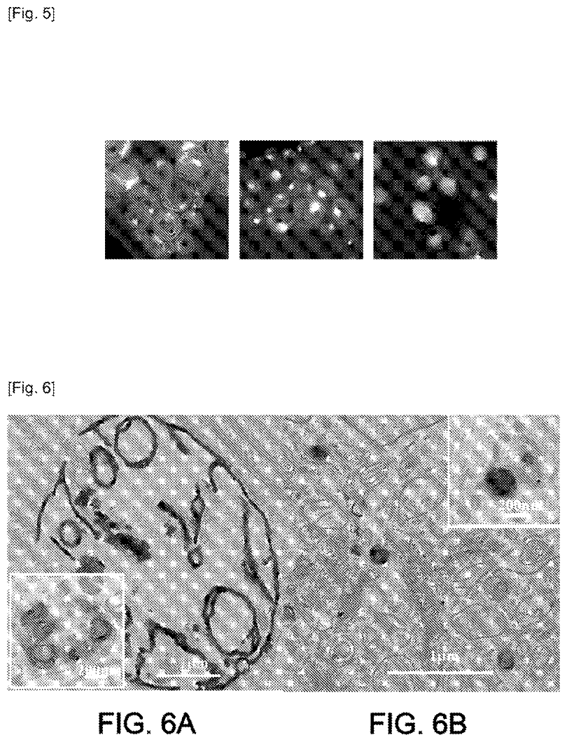

A third self-assembled structure used according to the invention is in the form of an assembly of metal oxide nanocrystals. Preferably, the nanocrystals forming this assembly are in the form of a mixture with ferrites of chemical formula Fe₃O₄ and y-Fe₂O₃ as the basis. The nanocrystals used to obtain this structure are obtained according to the method of synthesis of type 1 iron oxide nanocrystals comprising octadecene (of 6.5 nm or 10 nm). This type of assembly is hereinafter referred to as structure C and is illustrated in FIG. 3. Structure C, having a size between 70 and 270 nm, preferably between 100 and 200 nm, exhibits a 3D architecture in the form of a capsule comprising a central volume surrounded by a hollow shell formed by the said nanocrystals. The said central volume is partially filled with type 1 iron oxide nanocrystals comprising a hydrocarbon such as octadecene. Inside the shell, the iron oxide nanocrystals exhibit a spherical (3D) crystalline-type architecture within which the nanocrystals are organized in a face-centered cubic configuration.

Compared to structures A, structures C differ in that an excess of constituent nanocrystals is used during their synthesis. The filling rate of the central volume depends on the quantity of nanocrystals added in excess. The larger this quantity, the greater the filling rate will likewise be. A complete filling is, however, not sought.

The synthesis of such structures C is described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500. A detailed example is given below.

### Self-Assembled Structure of Type D

A fourth self-assembled structure used according to the invention is in the form of an assembly made starting from metal nanocrystals and metal oxides.

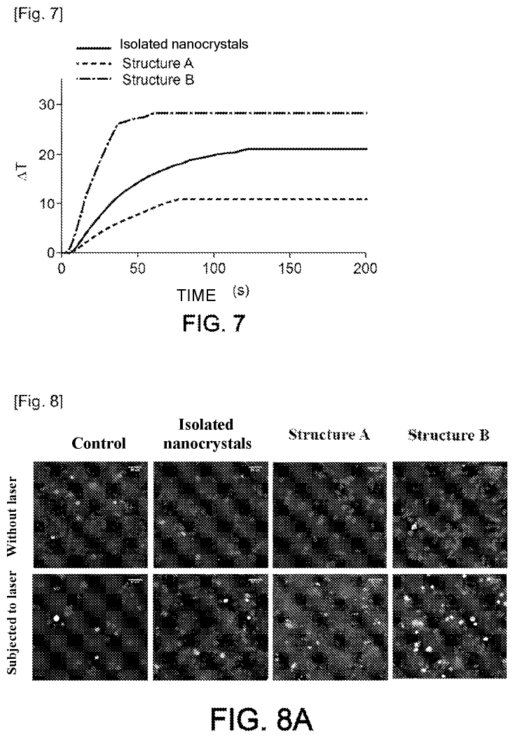

Preferably, the nanocrystals forming this assembly are in the form of a mixture with ferrites of chemical formula Fe₃O₄ and y-Fe₂O₃ as the basis and the metal nanocrystals are gold nanocrystals. The nanocrystals used to obtain this structure are obtained according to the methods of synthesizing 3.5 nm gold nanocrystals and synthesizing 6.5 nm type 1 iron oxide nanocrystals comprising a hydrocarbon, such as octadecene. This type of assembly is hereinafter referred to as structure D and is illustrated in FIG. 4. Structure D having a size between 70 and 270 nm, preferably between 100 and 200 nm, exhibits a 3D architecture in the form of a central capsule surrounded by a hollow shell formed by the said type 1 iron oxide nanocrystals. The said central volume is partially filled with a mixture of gold nanocrystals and type 1 iron oxide nanocrystals comprising a hydrocarbon, such as octadecene. Inside the shell, the nanocrystals exhibit a 3D cubic architecture of the NaZn₁₃— type, which is to say (Fe₃O₄)Au₁₃.

Compared to the structures C, the structures D differ in that they include gold in excess.

The synthesis of such structures D is described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500. A detailed example is given below.

### Self-Assembled Structure of Type E

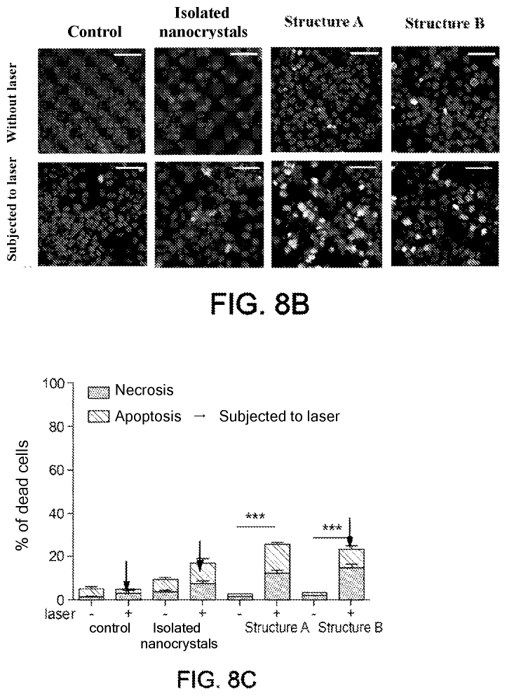

A fifth self-assembled structure used according to the invention is in the form of an assembly of metal nanocrystals and metal oxides. Preferably, the metal oxide nanocrystals forming this assembly are in the form of a mixture with ferrites of chemical formula Fe₃O₄ and y-Fe₂O₃ as the basis and the metal nanocrystals are gold nanocrystals. The nanocrystals used to obtain this structure are obtained according to the methods of synthesizing 5 nm gold nanocrystals and synthesizing 10 nm type 1 iron oxide nanocrystals comprising a hydrocarbon, such as octadecene. This type of assembly is hereinafter called structure E and is illustrated in FIG. 5. Structure E having a size between 50 and 100 nm exhibits a 3D architecture in the form of a capsule comprising a central volume surrounded by a hollow shell formed by the said type 1 iron oxide nanocrystals. The said central volume is partially filled with gold nanocrystals. Inside the shell, the gold nanocrystals present a 3D architecture of crystalline type within which the nanocrystals are organized in a face-centered cubic configuration.

Compared to structures D, structures E differ in that the central volume is filled exclusively with gold nanocrystals and not with a mixture of gold nanocrystals with type 1 iron oxide nanocrystals.

Such structures E are described in Yang, Z. et al; Supracrystalline colloidal eggs: epitaxial growth and freestanding three-dimensional supracrystals in nanoscaled Colloidosomes. Journal of the American Chemical Society 2016, 138, 3493-3500. A detailed example is given below.

### Synthesis of Structures A-E Example 1: Synthesis of a Structure A

3 mg of 6.5 nm type 1 iron oxide nanocrystals are dispersed in a solvent, the said solvent containing 200 μL chloroform and 8 μL octadecene. The obtained mixture is added to an aqueous solution containing 18 mg of dodecyltrimethylammonium bromide (DTAB). The obtained emulsion is vigorously stirred using the vortex for 30 seconds. Then, 5 mL of ethylene glycol solution containing 0.4 g of polyvinylpyrrolidone (PVP) (Mw=40,000 g/mol) is slowly added to the said emulsion, the mixture then being stirred with the vortex for 30 seconds. The emulsion thus obtained is heated to 70° C. under nitrogen and kept at this temperature for 15 minutes in order to have the internal chloroform phase evaporate. The obtained suspension was allowed to cool to room temperature. The assembled structures were washed twice with ethanol and then redispersed in deionized water.

The same modus operandi is used when the type 1 iron oxide nanocrystals used are 10 nm in size.

### Example 2: Synthesis of a Structure B

The process is that of example 1, with the exception that the starting nanocrystals are type 2 iron oxide nanocrystals and that the octadecene is not added during the said synthesis.

The same modus operandi is used when the type 2 iron oxide nanocrystals that are used are 10 nm in size.

### Example 4: Synthesis of a Structure C

3 mg of type 1 iron oxide nanocrystals are dispersed in a solvent, the said solvent containing 200 μL of chloroform and 8 μL of octadecene. The obtained mixture is added to an aqueous solution containing 18 mg of DTAB. The obtained emulsion is vigorously stirred with the vortex for 30 seconds. Then, 5 mL of ethylene glycol solution containing 0.4 g of PVP (Mw=40,000 g/mol) are added slowly into the said emulsion, the mixture is then stirred with the vortex for 30 seconds. The thus obtained emulsion is heated to 70° C. under nitrogen and kept at this temperature for 15 minutes in order to have the internal chloroform phase evaporate. The obtained suspension was allowed to cool to room temperature. The assembled structures were washed twice with ethanol and then redispersed in deionized water.

The same modus operandi is used when the type 1 iron oxide nanocrystals that are used are 10 nm in size.

### Example 5: Synthesis of a Structure D

3 mg of 6.5 nm type 1 iron oxide nanocrystals and 1 mg of 3.5 nm gold nanocrystals are dispersed in a solvent, the said solvent containing 300 μL of chloroform and 8 μL of octadecene. The obtained mixture is added to an aqueous solution containing 18 mg of DTAB. The obtained emulsion is vigorously stirred with the vortex for 30 seconds. Then, 5 mL of ethylene glycol solution containing 0.4 g of PVP (Mw=40,000 g/mol) is added slowly into the said emulsion, the mixture then being stirred with the vortex for 30 seconds. The obtained emulsion is heated to 70° C. under nitrogen and kept at this temperature for 15 minutes in order to have the internal chloroform phase evaporate. The obtained suspension was allowed to cool to room temperature. The assembled structures were washed twice with ethanol and then redispersed in deionized water.

### Example 5: Synthesis of a Structure E

Same protocol as that of example 4, apart from the starting quantities where 2 mg/L of 10 nm type 1 iron oxide nanocrystals and a quantity ranging from 0.2 mg/L to 2 mg/L of 5 nm gold nanocrystals are dispersed in a solvent, the said solvent containing 300 μL of chloroform and 8 μL of octadecene. The obtained mixture is processed as described previously. The filling rate of the central volume depends on the quantity of gold nanocrystals added. The larger this quantity, the greater the filling rate will likewise be. A complete filling is, however, not sought.

### Material and Measurement Methods

The compounds used are: iron (III) chloride hexahydrate (Sigma-Aldrich, 97%), oleic acid (Sigma-Aldrich, >90%), chloroform (Sigma-Aldrich, ≥ 99.5%), isopropyl alcohol (Aldrich, ≥ 99.7%), hexane (Sigma-Aldrich, 95%), toluene (Sigma-Aldrich, 99.8%), anhydrous ethanol (VWR, 99%), ethylene glycol (Sigma-Aldrich, 99.8%), dodecanethiol (Aldrich, ≥98%), 1-octadecene (Aldrich, 90%), dioctyl ether (Aldrich, 99%), dodecyltrimethylammonium bromide (TCI, >98%), dodecane (Sigma-Aldrich, ≥99%), chlorotriphenylphosphine Au (I) (Strem, 99.9%), borane tert-butylamine complex (Aldrich, 97%), Polyvinylpyrrolidone (PVP40, Sigma-Aldrich).

PBS (phosphate buffered saline t) is a phosphate saline solution buffer containing 137 mM NaCl₂, 7 mM KCl, 10 mM Na₂HPO₄, 1.76 mM KH₂PO₄.

Roswell Park Memorial Institute medium (0% bovine serum and 1% penicillin, with the whole kept at 37° C. under 5% CO₂ flow) is a well-known cell culture medium containing a large quantity of phosphates and is formulated for use in an atmosphere comprising 5% carbon dioxide.

A type A431 human epidermoid carcinoma cell line was studied.

Type A431 human epidermoid carcinoma cancerous cells (ATCC, CRL-1555) were grown in Roswell Park Memorial Institute medium (RPMI) supplemented with 10% fetal bovine serum and 1% penicillin and kept at 37° C. under 5% CO₂ flow.

The sizes of the self-assembled structures were measured from transmission electron microscopy (TEM) images obtained on a JEOL 1011 microscope at 100 kV. Thus, after synthesis, the structures are dispersed in water. A drop of the obtained solution, called “colloidal” is deposited on a copper grid upon which carbon has been deposited (called “TEM grid”). The images of these structures (minimum 500 structures) are taken after evaporation of the water. The size is measured thanks to the contrast observed on such an image. The diameter of each structure is measured at a given magnification. This measurement is made on between 100 and 200 structures. Using these obtained values, a histogram is drawn as a function of the different sizes measured. In general, a Gauss curve is obtained. The value corresponding to the maximum of this curve is retained.

The size distribution has been measured from the images obtained, as previously mentioned, on which a visual count has been made on between 100 and 200 structures. Starting from the histogram mentioned above, the distribution is deduced for the half-height deviation. This method provides a maximum value of the distribution. This counting can also be performed using software to measure the difference in contrast between the cells and the structures.

### EXAMPLES

### Application Example 1: Use in the Treatment of Cancer in a Mammal

The following results show the effectiveness of the use of self-assembled structures according to the invention which leads to the cellular destruction of cancerous cells of a tumor.

### Internalization and Maintenance of Self-Assembled Structures in Cancerous Cells

A comparative study was performed on the self-assembled structures A and structures B respectively prepared in examples 2 and 3, and the same unassembled metal oxide nanocrystals in order to analyze their ability to be internalized and how they are distributed inside cancerous cells. These systems are dispersed in water.

### Modus Operandi

The TEM images are obtained using a Hitachi HT7700 operating at 80 kV (MIMA2 platform, INRA, Jouy-en-josas, France). The A431 cells were exposed to unassembled Fe₃O₄ nanocrystals and to self-assembled structures A and structures B at 28 μg/mL of iron for 24 hours at 37° C. Following incubation, the cells were detached by trypsinization, washed twice with PBS and fixed with 2% glutaraldehyde in 0.1 M Na pH 7.2 cacodylate buffer for one hour at room temperature. The thus obtained samples were placed in contact with 0.5% Oolong Tea Extract (OTE) contrast agent in cacodylate buffer for one hour at room temperature, post-fixed with 1% osmium tetroxide containing 1.5% potassium cyanoferrate for one hour at room temperature, progressively dehydrated in ethanol: 70%-5 min incubation, 90%-5 min incubation, 100%-5 min incubation 3 times. The ethanol was progressively substituted by an ethanol-Epon mixture under vacuum for one hour and then incorporated into an Epon resin (Delta microscopy—Labège France).

Thin sections (70 nm) were collected on 200 copper grids and stained with lead citrate.

The grids were examined with a Hitachi HT7700 electron microscope operating at 80 kV (Elexience—France), and the images were acquired with a charge-coupled device camera (AMT).

The cells are incubated with either structures A or structures B. The incubated iron concentration is 28 μg/mL for 1 day at 37° C. under 5% CO₂ atmosphere. The cells are then washed with phosphate buffer saline (PBS) and then detached by trypsinization and redeposited in a Roswell Park Memorial Institute medium (RPMI).

The cells are introduced into a quartz chamber and subjected to a magnetic field gradient of 17 T/m induced by a permanent magnet B=0.15 T. The displacement of each cell is recorded using a video microscope in order to determine the velocity of the cell relative to the magnet in the steady state in which the magnetic force is compensated by the viscosity in the cell. For each condition, between 40 and 50 individual cells were recorded. The calculation of the iron concentration is deduced for each cell velocity. The magnetic moment can be translated into internalized mass by assuming a constant magnetic moment of 50 emu/gr for a magnetic field of 0.15 T.

### Results

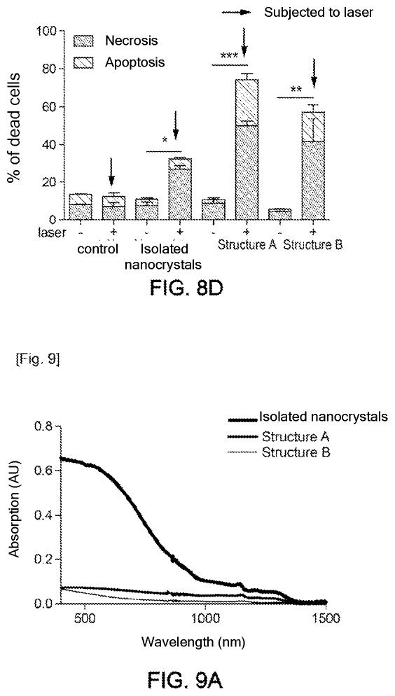

The images obtained are shown in FIG. 6.

In cancerous cells (A 431), both structures A and structures B are maintained. The size of the structures A changes considerably (up to a factor of 10) when they are internalized.

Starting from the transmission electron microscopy images, it is possible to identify the lysosomal membrane and to localize the structures due to the differences in contrast. The surface density of the nanocrystals in the lysosomes, corresponding to the ratio of the surface occupied by the nanocrystals to that of the lysosome, respectively reaches 34% and 52% for structures A and structures B, whereas it is only 4%. FIG. 6 (right) clearly shows that the contrasts are very localized. The percentage of nanocrystals in the proximity of the lysosomal surface (up to 100 nm) was measured. After one day of incubation, the surface density in proximity of the membrane is 10.6 times greater for structures A and 12.4 times greater for structures B when compared to the same nanocrystals that are isolated. Moreover, the quantity of iron was measured by magnetophoresis. Using magnetophoresis, the quantity of Fe expressed in picogram per cell (pg/cell) is between 2-14 pg/cell for the unassembled nanocrystals, between 2-22 pg/cell for structures A and between 2-20 pg/cell for structures B. The number of measurements is between 40 and 50. The ferrous incorporation per cell, on average, is 5.7 pg/cell for unassembled nanocrystals, 112 pg/cell for structures A and 9.25 pg/cell for structures B. This measurement is macroscopic.

These nanocrystals therefore concentrate in the proximity of the lysosomal membrane. If these assemblies are subjected to a magnetic field, they follow the orientation of the magnetic field. Such phenomena have already been observed with nanomaterials with well-known magnetic properties. In the present case, the nanocrystals used in unassembled state are not sensitive to the magnetic field whereas structures A and structures B are sensitive. This is again due to the fact that the nanocrystal assemblies are kept in the cells. The cells are incubated for a variable time period (between 4 hours and 8 days). Slices are then made and electron microscopy images are obtained in the various conditions. The nanocrystals are then observed by difference in contrast. The images show that the structures A are very strongly modified. Indeed, we lose the spherical structures observed in A. The structures are elongated/spherical. The size of the assemblies increases very noticeably. This is illustrated in FIG. 1 and in FIG. 6 (left). This will impact the collective magnetic properties in the cells (and therefore the magnetic effects observed). As regards the structures B, there is little variance in the size of the structures (FIG. 2 and FIG. 6 right). The spherical aspect is preserved.

It is therefore possible to localize the self-assembled structures in a precise location in the mammal in order to treat only the tissues affected by the disease and thus spare the healthy tissues thanks to their magnetic properties.

### Application Example 2: Hyperthermia in Cancerous Cells—Increase in the Temperature

The hyperthermia observed in type A431 cancerous cells was studied by comparing the self-assembled structures A and structures B to the unorganized nanocrystals dispersed in water.

### Irradiation of the Cells

Photo-thermal measurements were performed on cells in which structures A, structures B and unassembled nanocrystals were internalized for 24 hours and tested with the same quantity of Fe and subjected to laser irradiation for 5 min at 808 nm with an adjustable power (0-5 W) near infrared laser (NIR), Laser Diode Drivers, BWT). 100 μL of the aqueous dispersions of the self-assembled structures and of the unassembled nanocrystals were put in a 0.5 mL tube (open lid). The tube was irradiated at a distance of 4 cm with a laser spot of 0.4 cm². The laser power was adjusted to 1 W/cm². Temperature was monitored in real time using an infrared thermal camera (FLIR SC7000), recording one image per second. The temperature change (ΔT) was measured.

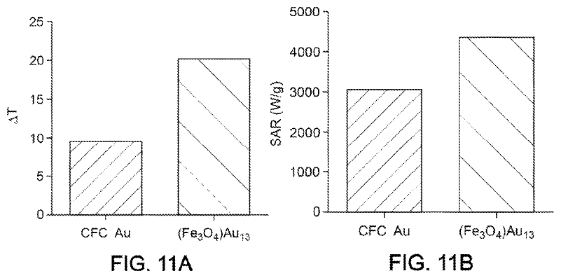

The results are presented in FIG. 9.

Thus, taking into account the local heat release induced by the light irradiation of the studied samples, it is possible to modulate the quantity of heat released by following the architecture of the self-assembled structure. Thus, the observed thermal behaviors are those where there is a greater increase of the temperature with structures B than with isolated nanocrystals. The addition of the structures B should therefore accentuate and consequently increase the local death of the cells of cancerous cells.

### Application Example 3. Hyperthermia in Cancerous Cells—Necrosis and Apoptosis

Hereafter, the local effects of necrosis and apoptosis of the cells linked to assembled or unassembled nanomaterials are studied. The level of cell necrosis and apoptosis is detected by the presence of several dyes: Hoechst 33342 (blue) detects living cells whereas YO-PRO (green) and propidium iodide (orangish red) detect cells that are necrotic (accidental cell death) or under apoptosis (programmed cell death). The major difference between YO-PRO and propidium iodide is related to the ability to penetrate cells: YO-PRO enters cells undergoing apoptosis whereas propidium iodide does not penetrate.

Modus operandi—Overall Cell Necrosis Analysis

A431 cells were incubated with unassembled [Fe] 28 μg/ml Fe₃O₄ nanocrystals, self-assembled structures for 24 hours in 6-well cell culture trays. Cells were treated with trypsin, washed with PBS once and counted. 1×10⁶ cells were suspended in 100 μl 1×PBS (500 μl tube). The cell suspension was irradiated with a laser beam at 808 nm for 10 minutes (1 W/cm², height 4 cm from the liquid/air interface, Laser Diode Drivers, BWT), and the temperature was recorded with an SC7000 infrared camera from FLIR Systems. In order to assess the overall effects of heating, the irradiated cells were put back into Nunc LabTeck plates (Thermo Fisher) and cultured for 4 hours. To test the level of cellular destruction exerted by the nano objects, the level of cellular necrosis was assessed. The kit called “Chromatin Condensation & Membrane Permeability Dead Cell Apoptosis Kit” from Molecular Probes-Invitrogen Detection Technologies (Eugene, OR) was used following the instructions provided by the kit manufacturer. In summary, the cells washed with PBS were incubated with 1 μl each of Hoechst 33342, YO-PRO staining, and propidium iodide at 37° C. in a 5% CO₂ atmosphere for 15 minutes. Cells were visualized using an Olympus JX81/BX61/Yokogawa CSU spinning disk confocal microscope (Andor Technology plc, Belfast) using the appropriate filters. The dead cells were stained with red-fluorescent propidium iodide whereas YO-PRO enters the cells in apoptosis, blue-fluorescent Hoechst 33342 stains the chromatin of the cells. 30 images were taken for each step. All experiments were performed three times. In order to quantify the level of necrosis and apoptosis, the number of nuclei stained respectively with propidium iodide, with YO-PR, as well as with Hoechst 33342 were counted using an ImageJ Cell Counter plugin.

### Modus Operandi—Cell Necrosis Analysis—Local

15,000 A431 cells were seeded in each well (1-18) in 18-well microplates (Ibidi, Germany). The cells were incubated with unassembled Fe₃O₄ nanocrystals, assembled structures at 28 μg/mL iron overnight and irradiated with a laser beam at 808 nm for 10 minutes (1 W/cm², height 4 cm from the liquid/air interface, Laser Diode Drivers, BWT). The cells were then incubated at 37° C., 5% CO₂ for 4 hours. Then, the level of cell destruction was tested using the Chromatin Condensation & Membrane Permeability Dead Cell Apoptosis Kit from Molecular Probes-Invitrogen Detection Technologies (Eugene, OR) following the protocol described above. Likewise, cell counting was performed in the same manner as described above.

Each of the structures was once again incubated with A431 cells at a fixed Fe concentration (28 mg/mL). After incubation, the cells are washed to remove nonincubated particles. One million incubated cells are mixed and suspended in 100 □L of solution. The increase in temperature is measured using a thermal camera. It can be seen that although the quantity of internalized iron is greater in structures A and structures B, the thermal effect is substantially the same as in the absence of cells. Only an increase in temperature of 1.7° C. and 3.3° C. for structures A and structures B is observed. In order to show that the damage created is local and not overall, two avenues are chosen: (1) the incubated and mixed cells are once again placed in a culture medium for 4 hours in order to assess the death of the cells; (2) in order to show the importance of the thermal effect, monolayers of internalized cells are irradiated. The experimental conditions remaining the same, the analysis is done 4 hours after irradiation.

### Results

Two types of experiments were performed to demonstrate the local effect of necrosis and apoptosis on the cancerous cells. An overall irradiation of the cell does not provide noticeable differences between the self-assembled structures and the isolated nanocrystals.

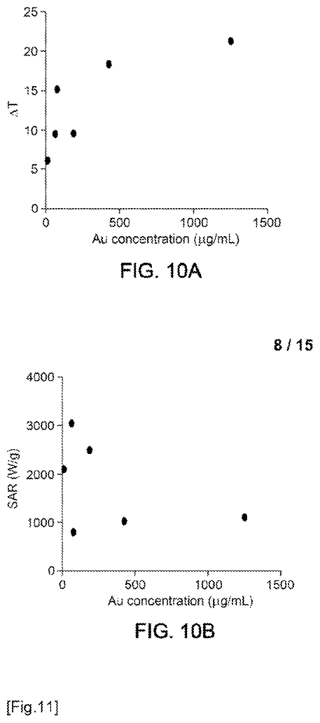

According to the first avenue (FIG. 8A), an increase in the death of the cells for structures A and structures B is shown in comparison to isolated nanocrystals. The level of apoptosis and necrosis is greater for structures B than for structures A than for isolated nanocrystals. According to the second avenue (FIG. 8B), a very important increase of the death of the cells is shown. This effect is greater for structures A than for structures B and the whole of structures A and structures B are much more effective than isolated nanocrystals.

The comparison of FIG. 8A and FIG. 8B clearly shows a considerable increase in necrosis and apoptosis in the second experiment in comparison to the first. This proves the importance of the local effect on the death of the cells.

### Application Example 4. Use in the Treatment of Rheumatism or in the Treatment of Cardiovascular Disease in a Mammal

As a function of the different types of self-assembly structures, it is possible to control the temperature of the heat release in order to reduce rheumatic pain.

The increase in the induced temperature was studied by subjecting aqueous solutions containing the dispersed self-assembled structures and unassembled nanocrystals to laser irradiation with a frequency in the near-infrared range (808 nm) and a power of 1 W/cm². The iron concentration of the solutions of the isolated oxide nanocrystals or in structures A and structures B is constant (1 mg/mL). The increase in the temperature induced by irradiation is recorded via a thermal camera. FIG. 9A shows that the absorption spectra of structures A and structures B are very weak compared to the same unassembled nanocrystals dispersed in water. Thus, the number of photons absorbed by structures A and structures B is much less than that absorbed by dispersed nanocrystals and thus a much lower temperature rise is expected. Despite this drawback, FIG. 9B shows that structure B induces a higher temperature rise than dispersed nanocrystals. This supports the “nano-radiator” effect brought about by the assembly.

The values given in these figures correspond to those obtained when the plateau is reached.

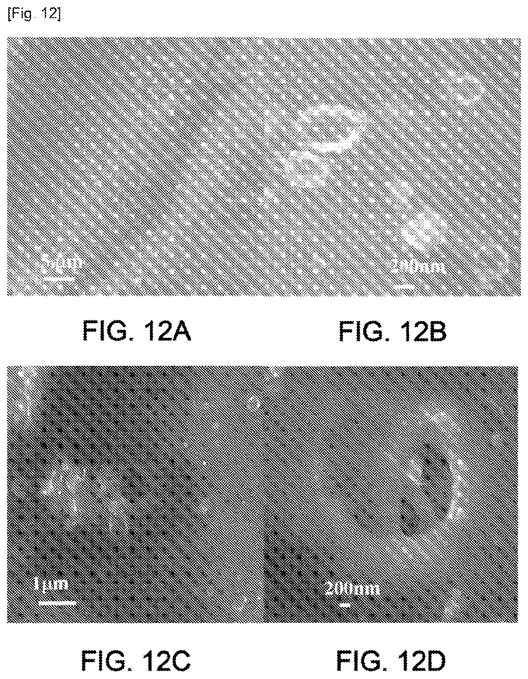

Moreover, the thermal effect has been compared for the same quantity of iron, of the structures of type E containing varying quantities of gold. FIG. 10 shows a significant thermal increase induced by laser irradiation of the structures of type C as a function of the quantity of gold present as part of the assembly. This thermal increase (FIG. 10A) is greater than in the absence of gold (which is to say, structure A). Moreover, the slope at the origin as a function of the irradiation time shows that the effect of the temperature is all the more important as the quantity of assembled gold nanocrystals forming the structure of type C is relatively low (FIG. 10B). In conclusion, depending on the quantity of gold present in the structure of type C, it is possible to control the temperature released.

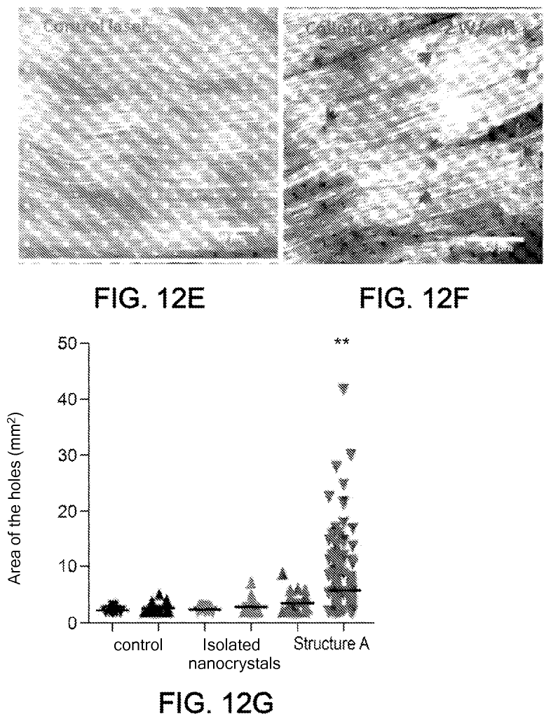

Let us recall that these structures are either made up of gold nanocrystals self-assembled according to a face-centered cubic lattice or in the form of a binary crystalline assembly of NaZn₁₃ type, which is to say, (Fe₃O₄)Au₁₃ (corresponding to the structure D of example 4). FIG. 11 shows that the structure containing a (Fe₃O₄)Au₁₃ type assembly is more efficient than the gold nanocrystals. This is more striking on the rise of the temperature (FIG. 11A) than on the slope at the origin, which is to say, the rate of increase in the temperature (FIG. 11B).

Whatever the assembly, the quantity of gold is constant, (4 mg/ml).

The whole of these results in liquid phase shows that, from one assembly to another, the temperature release can be controlled. This is useful for therapeutic applications in rheumatology where the quantity of heat released must not be such that the cells are necrotic or enter apoptosis.

### Application Example 5: Tests on Tendons

Let us recall that the tendon is made up of collagen fibers organized parallel to its longitudinal axis.

### Modus Operandi

Fresh sheep tendon was obtained from a butcher. The tendon was cut perpendicular to the fiber orientation using a scalpel into 1-2 cm fragments, which fragments were placed in the presence of 5% low-gelling temperature agarose (type VII-A; Sigma-Aldrich) prepared in PBS. Slices (100 μm) were cut parallel to the fiber direction with a vibratome (VT 1000S; Leica) in an ice-cold PBS bath. The slices were therefore placed on top of 0.4 μm organotypic culture insert (Millicell, Millipore) in 35 mm Petri dishes containing 1.1 mL RPMI 1640 without Phenol Red. [Fe] 100 μg of unassembled Fe₃O₄ nanocrystals on the one hand and self-assembled structures on the other hand were deposited on the slices. The said slices were incubated for 2 hours at 37° C. to allow the nanostructures to penetrate the tissue. The slices were washed in order to remove that which did not penetrate. The slices were irradiated with an 808 nm laser beam for 10 minutes (2 W/cm², height 4 cm of the slice, Laser Diode Drivers, BWT).

In order to evaluate the effects brought about on the collagen network, scanning electron microscopy images were obtained. After irradiation of the collagen slices, the slices were deposited directly on an electroconductive carbon tape with two adhesive sides. The samples were coated with carbon before observation. The images were obtained using a Zeiss Merlin Compact microscope at 10 kV.

### Results

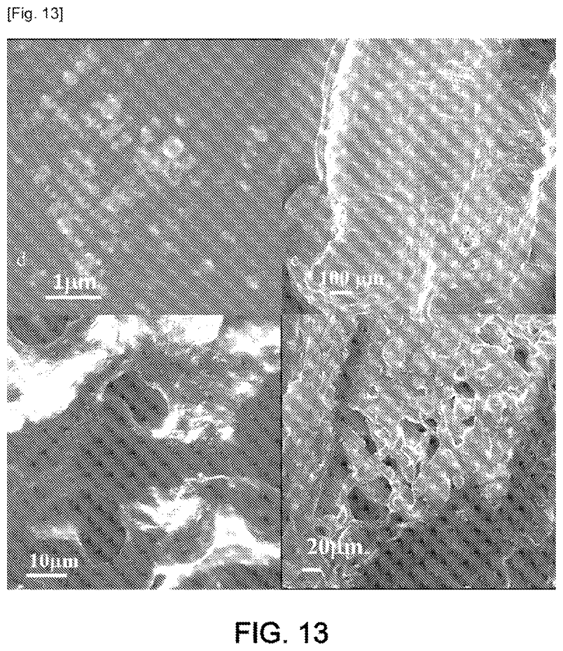

Depositions of structure A on a tendon followed by laser irradiation for 5 min led to destruction of the collagen with formation of “holes”. FIG. 12A and FIG. 12B show that the structures A are well localized on the tendon before irradiation. In addition, their size increased, again indicating excellent adhesion to the tendon. Using irradiation (FIG. 12C and FIG. 12D), we see the formation of “holes” indicating a denaturation of the collagen of the tendon. It was also possible to observe the formation of these “holes” by both electron and optical microscopy and the average area of these “holes” was able to be evaluated (FIG. 12G). This result therefore shows the usefulness of self-assembled structures in the destruction of cardiovascular attachments.

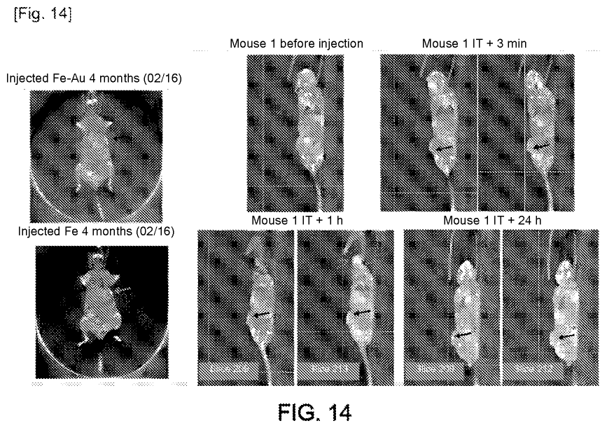

During the deposition of structures B, “holes” are observed by electron microscopy, whereas they were not observed by optical microscopy (FIG. 13). This could be due to the fact that the structures B are more rigid, inducing a less efficient internalization.

### Application Example 6: Assay on Collagen of the Extracellular Matrices of Cells

The effects induced by the presence of unassembled nanocrystals, of the structures B and structures A were studied on extracellular cell matrices in order to adapt the most appropriate self-assembled structures to the desired level of localized hyperthermia, either for therapeutic applications in rheumatology (application example 4) or for the destruction of cardiovascular attachments (application example 5).

### Modus Operandi

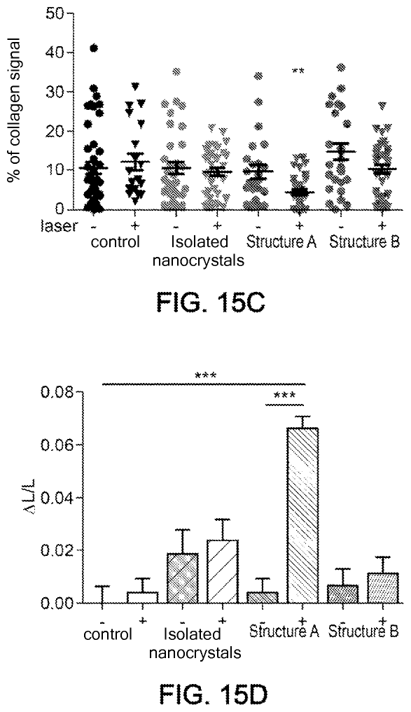

For this purpose, fresh ex vivo tumor slices of 350 μm thickness were incubated for 2 hours with unassembled ferrite nanocrystals, structures B and structures A. These slices were then exposed to a laser beam (808 nm at 2 W/cm²). To perform this assay, the EGI-1 tumor model was chosen, corresponding to a human desmoplastic cholangiocarcinoma. This one presents a greater quantity of extracellular matrix and a linear organization of collagen fibers that can be studied and quantified by two-photon fluorescence microscopy (TPF)—second harmony generation (SHG). The statistical results were obtained with One-way ANOVA, Kruskal-Wallis test with p<0.01 (FIG. 15A) and p<0.001 (FIG. 15B).

### Results

The SHG signal highlighting the presence of collagen was significantly reduced during the irradiation of the tumor slices incubated with structures D when compared to non-irradiated slices (control). Such a reduction was not observed in the case of tumor slices incubated with unassembled ferrite nanocrystals or structures B (FIG. 15A.) Analysis of the SHG images thus obtained were processed with CT-FIRE software also showed a significant decrease in collagen fiber length in the case of tumors incubated with the structures D and irradiated (FIG. 15B.) This may be due to the denaturation of the fibers caused by the local heating of the said structures D. Structures containing gold nanocrystals are therefore more efficient.

### Application Example 7. Use in the Diagnosis in a Mammal of Cancer by Medical Imaging

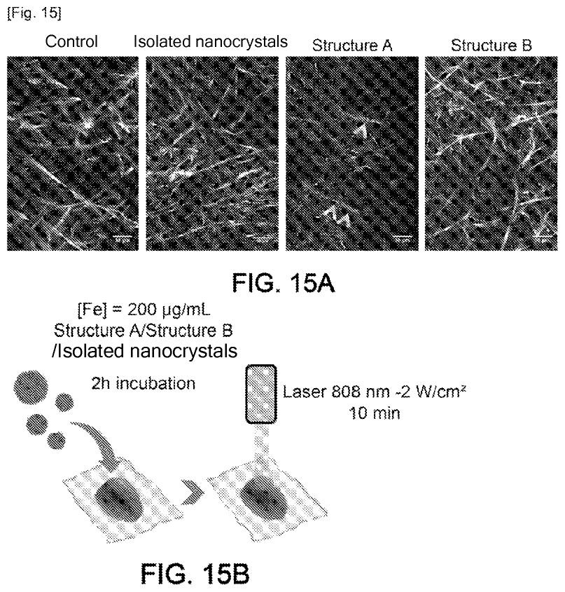

The results shown in FIG. 14 indicate that these assemblies can be used as contrast agents for computed tomography (CT) or magnetic resonance imaging (MRI).

These assemblies can be used to locate an area to be operated on.

Injection of MET-1 mice with self-assembled structures of type D does not cause any change in behavior compared to untested animals. Moreover, it was observed that the assembly is maintained, over time, in the injected regions. These injections are either subcutaneous or intraperitoneal.

It is therefore possible to combine the localization of the structures after injection with the help of medical imaging and the induction of cellular destruction of the cells containing the structures and having been treated by photothermal therapy. The cumulative effect brought about by these imaging and therapy agents and the possibility of moving the assemblies under a magnetic field, which facilitates access to inaccessible areas of the body, thereby makes it possible to develop medicine that is personalized.

The invention is not limited to the examples described above, which are given only by way of illustration, but rather it includes all the variants that the person skilled in the art may envisage in the context of the protection that is sought.

### Application Example 8. In Situ Dissolution of Self-Assembled Structures

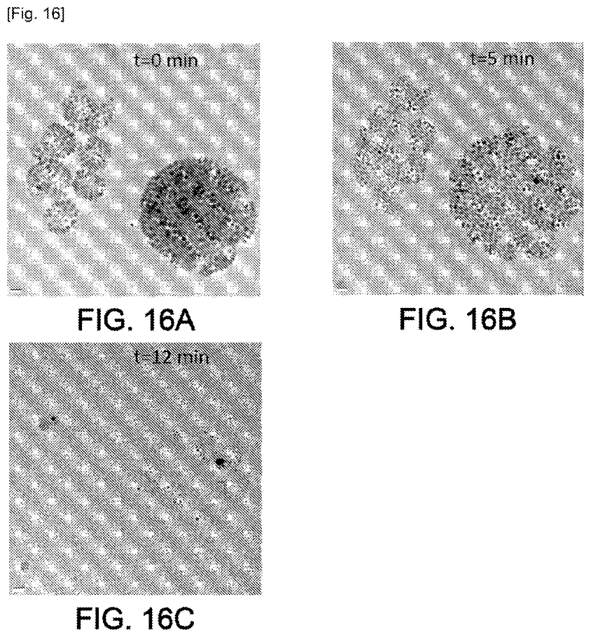

Under the effect of gamma radiation from the electron microscope at less than 200 keV, the dissolution of structures of type D is observed (FIG. 16). Indeed, a progressive dissolution of the self-assembled structures (FIG. 16A) is observed after 5 minutes of irradiation (FIG. 16B) all the way until the quasi-total disappearance after 12 minutes of irradiation (FIG. 16C).

Thus, following prolonged electron beam irradiation, the iron oxide nanocrystals become less and less visible until they are no longer detected. This is likely due to the deterioration of the carbon chains used to maintain the integrity of the self-assembled structures. The remaining gold nanocrystals tend to fuse.

This allows it to be concluded that the nanocrystals used to obtain the self-assembled structures dissolve under the action of an electron beam. The interest is great because this indicates that the self-assembled structures, instead of accumulating in the liver and other organs, can be dissolved under the action of an electron beam in the constituent nanocrystals.

1. A self-assembled structure for use in the treatment of mammalian cancer, the said structure having a size of 50 to 1,500 nm comprises a plurality of nanocrystals selected from metal or metal oxide nanocrystals, a plurality of ligands covalently bound to the surface of each of the said nanocrystals.

2. A structure according to claim 1, wherein the nanocrystals are selected from gold nanocrystals, iron oxide nanocrystals of the chemical formulae Fe₃O₄ and y-Fe₂O₃, or mixtures thereof.

3. The structure according to claim 1, wherein the ligands comprise hydrocarbon chains of 4 to 36 carbon atoms, or are of polymeric nature and are covalently bound to the surface of each of the nanocrystals by a functional group selected from sulfanyl, carboxyl and hydroxyl groups.

4. The structure according to claim 1, wherein the nanocrystals have a plurality of surfactants adsorbed on their surfaces, the said surfactants being selected from phospholipids, such as distearoylphosphatidylcholine, distearoylphosphatidylethanolamine, dipalmitoylphosphatidylcholine and dipalmitoylphosphatidylethanolamine; the halogenated cationic compounds having a chain comprising 12 to 16 carbon atoms such as cetylpyridinium chloride, cetylpyridinium bromide, cetyltrimethylammonium bromide, cetyltrimethylammonium chloride, cetyldimethylethylammonium bromide, cetylbenzyldimethylammonium chloride, cetyltributylphosphonium bromide, dodecyltrimethylammonium bromide and tetradecyltrimethylammonium bromide.

5. The structure according to claim 1, wherein the nanocrystals exhibit a plurality of aliphatic hydrocarbons adsorbed to their surfaces, the said aliphatic hydrocarbons being selected from hydrocarbons having from 16 to 36 carbon atoms.

6. The structure according to claim 1, the structure is wherein it is in the form of a capsule comprising a central volume surrounded by a hollow shell formed by nanocrystals; the said central volume being empty or partially filled by nanocrystals.

7. The structure according to claim 6 wherein the shell is formed by metal oxide nanocrystals and the central volume is partially filled with a mixture of metal nanocrystals and metal oxide nanocrystals.

8. The structure according to claim 1 comprising metal oxide nanocrystals, the structure is characterized in that it exhibits a crystalline-type architecture within which the nanocrystals are organized in a face-centered cubic configuration.

9. The structure according to claim 1, wherein the size of the nanocrystals is between 2 and 12 nm.

10. The structure according to claim 1, wherein the size distribution of the nanocrystals is less than 10%.

11. A composition in a form suitable for injection comprising self-assembled structures according to claim 1 and an excipient or saline solution.

12. The composition according to claim 12, wherein it is aqueous.

13-14. (canceled)

15. A method of inducing cellular destruction of cancerous cells in a mammal by photothermal and/or magnetothermal treatment comprising contacting the cancerous cells with a plurality of the self-assembled structures of claim 1 and irradiation thereof with infrared light, and/or respectively the application of a magnetic field.

16. A method of inducing cellular destruction of cancerous cells in a mammal by radiation therapy comprising contacting the cancerous cells with a plurality of the self-assembled structures of claim 1 and administration of a dose of radiation therapy.
